(19) United States
(12) Patent Application Publication
(10) Pub. No.: US 2025/0099408 A1
(43) Pub. Date: Mar. 27, 2025

(54) IMPROVED SURAMIN METHODS AND COMPOSITIONS

(71) Applicant: Sammy Oyoo OPIYO, Nairobi (KE)

(72) Inventor: Sammy Oyoo OPIYO, Nairobi (KE)

(21) Appl. No.: 18/730,814

(22) PCT Filed: Jan. 20, 2023
(86) PCT No.: PCT/KE2023/000001

§ 371 Date: Jul. 22, 2024

(30) Foreign Application Priority Data
Jan. 21, 2022 (KE) 2022/3986

(51) Int. Cl. A61K 31/185; A61K 45/06

(52) U.S. Cl. CPC A61K 31/185; A61K 45/06

(57) ABSTRACT

The invention disclosed herein are improved suramin-containing methods and pharmaceutical compositions.

### FIELD

Disclosed herein are improved suramin-containing methods and compositions.

### BACKGROUND

Suramin has been used in treating human trypanosomiasis for a long time. After the discovery of HIV in the early 1980s, the first product to be tried on this condition was suramin by the USA suramin working group in 1984. This was done directly without animal studies as the dosage was to be maintained at 1 g IV. The product did not give clinical improvements to the patients, despite almost clearing the virus from the blood circulation. This was attributed to the reservoir nature of the condition by hiding in the lymphatic system away from the poorly distributed suramin. In the other trials involving various cancer conditions, suramin has been tested, giving some results which were generally not very satisfactory.

Despite years of experience working with this compound, researchers have been unable to produce a formulation with clinical efficacy and an acceptable safety profile.

### SUMMARY

Provided herein are pharmaceutical compositions, comprising improved formulations of suramin.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise modified suramin formulations.

In some embodiments, there is provided a pharmaceutical composition comprising a lysine salt of suramin, formulated for sublingual administration. In other embodiments, the composition is formulated for buccal administration.

Reference herein to lysine salts, except where indicated otherwise, refers to salts with lysine or a derivative thereof, including, e.g., bis-lysine salts and mono-lysine salts. While the lysine may be in the L form, D form, or DL form, the L form is a particular example. In other embodiments, dipeptides containing lysine can be utilized. “Dipeptide” refers to an amino acid dimer wherein two amino acid molecules are peptide-bonded, and the “dipeptides containing lysine” can refer to a dipeptide wherein at least one molecule, or 2 molecules, of the dipeptide is lysine. Examples of the dipeptides containing lysine include lysyl-lysine and the like. In still other embodiments, oligopeptides, e.g. peptides having a length of 2-6 amino acids, or in other embodiments 2-5, 2-4, or 2-3 amino acids many be used, provided that the peptide comprises lysine.

In other embodiments, there is provided a pharmaceutical composition comprising an arginine salt of suramin, formulated for sublingual administration. In other embodiments, the composition is formulated for buccal administration.

Reference herein to arginine salts, except where indicated otherwise, refers to salts with arginine or a derivative thereof, including, e.g., bis-arginine salts and mono-arginine salts. Relevant forms of arginine include D,L-arginine, L-arginine, and alkyl (e.g. ethyl, methyl, propyl, isopropyl, butyl, isobutyl, t-butyl) esters of L-arginine. the L form is a particular example. In other embodiments, dipeptides containing arginine can be utilized. “Dipeptide” refers to an amino acid dimer wherein two amino acid molecules are peptide-bonded, and the “dipeptides containing arginine” can refer to a dipeptide wherein at least one molecule, or 2 molecules, of the dipeptide is arginine. In still other embodiments, oligopeptides, e.g. peptides having a length of 2-6 amino acids, or in other embodiments 2-5, 2-4, or 2-3 amino acids many be used, provided that the peptide comprises arginine.

Those skilled in the art will appreciate, in light of the present disclosure, that buccal and sublingual oral dosage forms are dosage forms that are intended to be held in the mouth or under the tongue until they have completely dissolved. Unlike most oral dosage forms in which the pharmaceutically active ingredient is delivered to the gastrointestinal tract of the patient for absorption of the drug through the stomach or intestinal epithelium, sublingual and/or buccal dosage forms are designed to release the pharmaceutically active ingredient in the mouth for absorption through oral mucosa. Buccal dosage forms are intended to be inserted into the buccal pouch (a space generally defined between a check and the gums) and dissolve or erode relatively slowly (e.g. in 5-15 minutes), whereas sublingual oral dosage forms are intended to be held under the tongue and dissolve more rapidly (e.g. in 2-5 minutes). As a result, buccal dosage forms, including mucoadhesive formulations, are generally formulated with excipients to optimize drug release into and through oral mucosa and to minimize release of the drug into the gastrointestinal tract, which is within the skill of those skilled in the art.

In other embodiments, compositions formulated for sublingual administration typically comprise a non-ionic polymeric solubility enhancer; a mucoadhesive polymer; a disintegrant; and/or a filler; see US 2017/0246158 to Mutasem Rawas-Qalaji et al., and US 2011/0028431 to Horst G Zerbe et al., which are incorporated herein by reference.

In certain embodiments, the described pharmaceutical compositions are indicated for administration to a human at a dose of 15-50 milligrams (mg) of the described suramin salt, or, in other embodiments, 15-45, 15-40, 15-35, 15-30, 20-50, 20-45, 20-40, 20-35, 20-30, 21-29, 22-28, 23-27, or 24-26 mg of the described suramin salt, which may be, in various embodiments, a lysine salt or an arginine salt. In further embodiments, the pharmaceutical composition is indicated for administration up to three times daily, or, in more specific embodiments, once, twice, three times, or 1-2 times daily. In other embodiments, the pharmaceutical composition is indicated for administration 2-4, 2-3, or 3-4 times daily. In certain embodiments, the daily dose does not exceed 50 mg, or, in other embodiments, 40, 35, 30, 25, 20, 15, or 10 mg. Alternatively or in addition, the composition is administered for a maximum length of 90 days, or in other embodiments, 80 days, 70 days, 60 days, 50 days, 40 days, or 30 days.

In still other embodiments, the pharmaceutical compositions are indicated for administration to administration to an adult human (e.g., weighing an average of 70 kg.) at a dose of 30-100 mg of the suramin salt, or, in other embodiments, 20-350, 20-50, 40-100, 30-80, 40-80, 50-100, 75-125, 100-150, 125-175, 150-200, 175-225, 200-250, 225-300, 200-300, 225-325, 250-350 mg of the suramin salt, which may be, in various embodiments, a lysine salt or an arginine salt. In further embodiments, the pharmaceutical composition is indicated for administration up to three times daily, or, in more specific embodiments, once, twice, three times, or 1-2 times daily. In other embodiments, the pharmaceutical composition is indicated for administration 2-4, 2-3, or 3-4 times daily. In certain embodiments, the daily dose does not exceed 350 mg, or, in other embodiments, 300, 250, 200, 150, 120, 100, 80, 70, or 60 mg. Alternatively or in addition, the composition is administered for a maximum length of 90 days, or in other embodiments, 80 days, 70 days, 60 days, 50 days, 40 days, or 30 days.

In still other embodiments, the described pharmaceutical composition is indicated for administration to a human at a dose of 0.3-0.6 mg/kg body weight; or, in other embodiments, 0.3-0.5, 0.35-0.45,.3-0.4, 0.4-0.5, or 0.4-0.6 mg/mg body weight of the suramin salt, which may be, in various embodiments, a lysine salt or an arginine salt. In further embodiments, the pharmaceutical composition is indicated for administration up to three times daily, or, in more specific embodiments, once, twice, three times, or 1-2 times daily. In other embodiments, the pharmaceutical composition is indicated for administration 2-4, 2-3, or 3-4 times daily. In yet other embodiments, the dosage is 0.3-5.0 mg/kg body weight, or, in more specific embodiments, 0.5-0.8, 0.6-1.0, 0.8-1.5, 1-1.5, 1-1.8, 1.2-1.8, 1.5-2.0, 2.0-2.5, 2.5-3.0, 3.0-4.0, 3.5-4.5, or 4-5 mg/kg body weight. In certain embodiments, the daily dose does not exceed 0.6 mg/kg body weight, or, in other embodiments, 0.5, 0.4, 0.3, 0.2, or 0.1 mg/kg body weight. Alternatively or in addition, the composition is administered for a maximum length of 90 days, or in other embodiments, 80 days, 70 days, 60 days, 50 days, 40 days, or 30 days.

In yet other embodiments, for short-term administration, 3.0-10.0 mg/kg body weight of the suramin salt (e.g. for a human subject) is administered. In other embodiments, the dose is 3.0-4.0, 3.5-4.5, 4.0-5.0, 4.5-5.5, 5-7, 6-8, or 7-10 mg/kg body weight. Short-term administration, as used herein, may refer to administration for up to 2 weeks, or, in other embodiments, up to 10 days, or up to 8, 7, 6, 5, 4, 3, 2, or 1 day.

In yet other embodiments, administration of the suramin salt is discontinued when a maximal tolerated body fluid concentration is reached. The maximal concentration may be, in some embodiments, 15 micromoles per liter (micromolar), or, in other embodiments, 20, 18, 12, 10, 8, 6, or 5 micromolar. Optionally, administration may be recontinued when the body fluid concentration drops, e.g. to 50% of the maximal tolerated body fluid concentration.

In still other embodiments, the described pharmaceutical composition is indicated for treatment of a virus selected from HIV/AIDS, Chikungunya, Ebolavirus, Hepatitis B virus, Zika virus, Hand Foot and Mouth Disease, each of which represents a separate embodiment. Those skilled in the art will appreciate that Hand Foot and Mouth Disease is often caused by Coxsackievirus A16 or enterovirus 71.

In still other embodiments, the described pharmaceutical composition is indicated for treatment of a coronavirus. In more specific embodiments, the coronavirus is COVID-19.

In other embodiments, the coronavirus described herein is human and bat severe acute respiratory syndrome coronavirus (SARS-CoV) of the type severe acute respiratory syndrome-related coronavirus, e.g. SARS-CoV-1 and SARS-CoV-2.

In other embodiments, the described virus is any virus in the family Coronaviridae, e.g. comprising Cornidovirineae (Orthocoronavirinae) and Letovirinae.

In other embodiments, the described virus is any virus in the suborder Cornidovirineae, e.g., comprising Alphacoronaviruses, Betacoronaviruses, Gammacoronaviruses, and Deltacoronaviruses

In other embodiments, the described virus is any virus in the order Nidovirales, e.g., comprising Cornidovirineae, Tornidovirineae, Mesnidovirineae, Ronidovirineae, Nanidovirineae, and Arnidovirineae.

In other embodiments, the described virus is any virus in the realm Riboviria. Taxonomy of coronaviruses is known to those skilled in the art, and is described, e.g., in Siddell, S G et al. 2019; Ziebuhr, J et al., 2017); Ziebuhr, J. et al., 2019); and Gorbalenya, S et al. (2020).

In certain embodiments, the treated virus is SARS-CoV-2, e.g. a virus having a sequence at least 96%, or in other embodiments, at least 91%, at least 92%, at least 93%, at least 94%, at least 95%, at least 96%, at least 97%, at least 98%, or at least 99% homologous to at least 1 sequence selected from the nucleotide sequences set forth in GenBank Accession Nos. NC_045512.2, MT126808, MT123290, MT093571, MT066176, MT263074, MT276331, MT233523, MT066156, and LC528233 (SEQ ID NOs. 1-10).

In other embodiments, the treated coronavirus has a sequence at least 96%, or in other embodiments, at least 91%, at least 92%, at least 93%, at least 94%, at least 95%, at least 96%, at least 97%, at least 98%, or at least 99% homologous to at least 1 sequence selected from the nucleotide sequences set forth in GenBank Accession Nos. NC_004718.3, AY274119.3, GU553363.1, DQ182595.1, AY297028.1, and AY515512.1 (SEQ ID NOs. 11-16).

In other embodiments, the treated virus is related to a bat coronavirus, e.g. related to GenBank Accession No. DQ022305 (SEQ ID NO. 17).

Alternatively or in addition, the pharmaceutical composition further comprises an antiretroviral compound. In certain embodiments, the antiretroviral compound is selected from saquinavir, indinavir, ritonavir, nelfinavir, amprenavir, fosamprenavir, lopinavir, atazanavir, tipranavir, and darunavir, each of which represents a separate embodiment.

In other embodiments, the antiretroviral compound is selected from a thymidine analogue, e.g. zidovudine (AZT) and stavudine (d4T); a cytidine analogue, e.g. zalcitabine (ddC), lamivudine (3TC), and emtricitabine (FTC); a guanosine analogue; e.g. abacavir (ABC) and entecavir (ETV); and an adenosine analogue, e.g. didanosine (ddI), tenofovir (TDF), and adefovir (ADV), each of which represents a separate embodiment.

In still other embodiments, the antiretroviral compound is another antiretroviral compound known in the art.

In still other embodiments, the described pharmaceutical composition is indicated for treating a Tuberculosis infection. In certain embodiments, the Tuberculosis strain is a multi-drug resistant strain.

In still other embodiments, the described pharmaceutical composition is indicated for treating an Onchocerciasis infection. Those skilled in the art will appreciate that a disease colloquially known as river blindness is caused by infection with the parasitic worm Onchocerca volvulus.

In still other embodiments, the described pharmaceutical composition is indicated for treatment of a neoplasia or malignancy. In certain embodiments, the malignancy is a leukemia.

In some embodiments, the leukemia is selected from Acute lymphoblastic leukemia, Chronic lymphocytic leukemia, Acute myelogenous leukemia, Chronic myelogenous leukemia, Hairy cell leukemia, T-cell prolymphocytic leukemia, and Juvenile myelomonocytic leukemia, each of which represents a separate embodiment. In other embodiments, the malignancy is a lymphoma. In some embodiments, the lymphoma is selected from Hodgkin lymphoma and non-Hodgkin lymphoma, which may be a B-cell lymphoma (e.g., Diffuse large B-cell lymphoma (DLBCL), Follicular lymphoma, Chronic lymphocytic leukemia (CLL)/Small lymphocytic lymphoma (SLL), Mantle cell lymphoma (MCL), Marginal zone lymphomas, Burkitt lymphoma, Hairy cell leukemia (which may be a lymphoma subtype), Waldenstrom macroglobulinemia, Primary central nervous system lymphoma, Primary eye (intraocular) lymphoma; or a T-cell lymphoma (e.g., Precursor T-lymphoblastic lymphoma, Cutaneous T-cell lymphoma (including mycosis fungoides and Sézary syndrome), or Adult T-cell leukemia/lymphoma), each of which represents a separate embodiment. In other embodiments, the malignancy is a myeloma. In some embodiments, the myeloma is selected from Multiple myeloma, Plasmacytoma, Localized myeloma, or Extramedullary myeloma, each of which represents a separate embodiment.

In still other embodiments, the described pharmaceutical composition is indicated for treatment of a prostate carcinoma. In more specific embodiments, the prostate carcinoma may be a hormone-refractory tumor. Alternatively or in addition, the pharmaceutical composition is a first-line treatment for the tumor.

In still other embodiments, the described pharmaceutical composition is indicated for prevention of metastases. In more specific embodiments, the composition is administered perioperatively, e.g. as a prophylaxis prior to a surgical intervention in cancer management, for example solid tumor surgical removal. A non-limiting list of tumors that are treated with surgical removal includes breast cancer, cervical cancer, colon cancer, and bone cancer (e.g. osteosarcoma). In certain embodiments, the composition is administered up to one month before surgery (or less, if the dictated by urgency of the procedure), continuing for up to one month afterwards. In other embodiments, the composition is administered from 1-3 weeks before the surgery through 1-3 weeks afterwards, or, in still other embodiments, from 2-3 weeks before the surgery through 1-2 weeks afterwards; or from 1-2 weeks before the surgery through 2-3 weeks afterwards. Method for surgical removal of tumors, and risks associated therewith, are known to those skilled in the art, and are described, for example, in Tohme S et al., Surgery for Cancer: A Trigger for Metastases. Cancer Res. 2017 Apr. 1; 77(7): 1548-155.

In still other embodiments, the described pharmaceutical composition is indicated for prevention of reduction of the incidence of development of cancer from a pre-cancerous growth. In more specific embodiments, the growth is inoperable. In other embodiments, the composition is administered perioperatively, e.g. as a prophylaxis prior to a surgical intervention to remove the growth. In more specific embodiments, the growth may be on the skin (e.g., actinic keratosis, Bowen's disease [intraepidermal carcinoma/squamous carcinoma in situ], or dyskeratosis congenita; in the breast (e.g., ductal carcinoma in situ, sclerosing adenosis, or small duct papilloma); in the head, neck, or oral cavity (e.g., oral submucous fibrosis, erythroplakia, lichen planus (oral), leukoplakia, proliferative verrucous leukoplakia, or stomatitis nicotina); gastrointestinal (e.g., Barrett's esophagus, atrophic gastritis, colon polyp, Plummer-Vinson syndrome (sideropenic dysphagia), or hereditary nonpolyposis colorectal cancer); gynecological (e.g., cervical dysplasia [cervical intraepithelial neoplasm, CIN], vaginal intraepithelial neoplasm [VAIN], anal dysplasia, lichen sclerosus, Bowen's disease [penile or vulvar], or erythroplasia of Queyrat); urological (e.g., bladder carcinoma in situ), or hematological (e.g., monoclonal gammopathy of unknown significance).

A non-limiting list of tumors that are prevented or impeded by the described methods and compositions includes breast cancer, cervical cancer, colon cancer, and bone cancer (e.g. osteosarcoma). In certain embodiments, the composition is administered up to one month before surgery (or less, if the dictated by urgency of the procedure), continuing for up to one month afterwards. In other embodiments, the composition is administered from 1-3 weeks before the surgery through 1-3 weeks afterwards, or, in still other embodiments, from 2-3 weeks before the surgery through 1-2 weeks afterwards; or from 1-2 weeks before the surgery through 2-3 weeks afterwards. Method for surgical removal of tumors, and risks associated therewith, are known to those skilled in the art, and are described, for example, in Tohme S et al., Surgery for Cancer: A Trigger for Metastases. Cancer Res. 2017 Apr. 1; 77(7): 1548-155.

In still other embodiments, the described pharmaceutical composition is indicated for treatment of a metastatic cancer, e.g. IGF (Insulin-like Growth Factor)-dependent solid and non-solid tumors. More specific embodiments of IGF-dependent solid tumors are IGF-1 dependent and IGF-2 dependent tumors of the prostate, breast, colorectal, and ovaries; Ewing's sarcoma; Glioblastoma multiforme (GBM); and lung cancer (e.g. non-small cell lung cancer [NSCLC], including the squamous cell subtype). In certain embodiments, the pharmaceutical composition is a first-line treatment for the tumor. Without wishing to be limited by theory, the described compositions may be useful for first-line treatment of e.g., IGF-dependent tumors, due to their effect on IGF.

In other embodiments, the pharmaceutical composition is indicated for prophylaxis or treatment of an IGF-1 dependent second primary cancer, meaning a malignant tumor diagnosed at the same time as the primary tumor or later, which is often in a different organ and is not a metastasis or recurrence of the original primary cancer. In more specific embodiments, the second primary cancer may be selected from breast cancer (e.g. following treatment for Hodgkin's lymphoma, for example with supradiaphragmatic irradiation); a secondary lung cancer (e.g. following treatment for a resected primary lung cancer, breast cancer, or and head and neck cancer); a bladder, renal, or endocrine cancer (e.g. following pelvic radiation therapy for prostate cancer); prostate cancer (e.g. following bladder cancer); or secondary colorectal cancer (e.g. following radiotherapy for prostate cancer or abdominopelvic radiation for cervical cancer).

In certain embodiments, the pharmaceutical composition further comprises a chemotherapeutic agent. In certain embodiments, the chemotherapeutic agent is selected from Vincristine (including liposomal vincristine), Daunorubicin (daunomycin), doxorubicin (Adriamycin), Cytarabine (e.g. cytosine arabinoside or ara-C), L-asparaginase or PEG-L-asparaginase (pegaspargase or Oncaspar); 6-mercaptopurine (6-MP), Methotrexate, Cyclophosphamide, or Prednisone. In other embodiments, the agent is selected from Cladribine (Leustatin, 2-CdA), Fludarabine (Fludara), Mitoxantrone, Etoposide (VP-16), 6-thioguanine (6-TG), Hydroxyurea, Corticosteroid drugs, such as prednisone or dexamethasone, Methotrexate (MTX), 6-mercaptopurine (6-MP), Azacitidine (Vidaza), and Decitabine (Dacogen).

In other embodiments, the chemotherapeutic agent is selected from imatinib mesylate, bortezomib, bicalutamid, gefitinib, and adriamycin; or in other embodiments alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trictylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside; cyclophosphamide; thiotepa; taxanes, e.g., paclitaxel and docetaxel; retinoic acid; esperamicins; capecitabine; and pharmaceutically acceptable salts, acids or derivatives of any of the above. In other embodiments, the agent is an anti-hormonal agent, such as anti-estrogens, including for example tamoxifen, Nolvadex™, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). In other embodiments, the agent is selected from anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antincoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl) amine, Troxacitabine, Uramustine, Vadimezan, Vinflunine, ZD6126 or Zosuquidar.

In still other embodiments, the described pharmaceutical composition is indicated for co-administration with cancer radiotherapy.

In still other embodiments, the described pharmaceutical composition is indicated for treatment of a diabetic nephropathy.

In still other embodiments, the described pharmaceutical composition is indicated for treatment of autism.

In other embodiments, the described pharmaceutical compositions comprise, in addition to the described suramin salts, an arginase inhibitor. In certain embodiments, the arginase inhibitor is present together with both a suramin salt and another compound mentioned herein. Non-limiting examples of arginase inhibitors include INCB1158; (3R,4S)-3-acetamido-4-allyl-N-(tert-butyl)pyrrolidine-3-carboxamide; AEB1102 (pegylated recombinant arginase, Aeglea Biotherapeutics); CB-1158 (Calithera Biosciences); piceatannol-3′-O-β-d-glucopyranoside (PG); and those described in US 2019/0054101 and US 2018/0360860, which are incorporated herein by reference. In still other embodiments, the arginase inhibitor is norNOHA (acetate) (N^{omega}-hydroxy-nor-L-arginine; CAS No. 1140844-63-8).

In still other embodiments, the dose of norNOHA acetate (e.g. for a human subject, who may be, in more specific embodiments, an adult human) is about 1-4 mg, or in other embodiments, 1-4, 1.3.5, 1-3, 1.5-3, 1.1-2.9, 1.2-2.8, 1.3-2.7, 1.4-2.6, 1.5-2.5, 1.6-2.4, 1.7-2.3. or 1.8-2.2 mg. In further embodiments, the pharmaceutical composition is indicated for administration up to three times daily, or, in more specific embodiments, once, twice, three times, or 1-2 times daily. In other embodiments, the pharmaceutical composition is indicated for administration 2-4, 2-3, or 3-4 times daily. In still further embodiments, the pharmaceutical composition further includes a lysine salt, or in other embodiments an arginine salt of suramin, at a dose of 15-50 mg, or, in other embodiments, 15-45, 15-40, 15-35, 15-30, 20-50, 20-45, 20-40, 20-35, 20-30, 21-29, 22-28, 23-27, or 24-26 mg. In certain embodiments, the daily dose does not exceed 25 mg, or, in other embodiments, 22, 20, 17, 15, 12, 10, 8, or 7 mg. Those skilled in the art can readily calculate the doses of other norNOHA salts from the described doses of norNOHA acetate. Alternatively or in addition, the composition is administered for a maximum length of 90 days, or in other embodiments, 80 days, 70 days, 60 days, 50 days, 40 days, or 30 days.

In still other embodiments, the dose of norNOHA acetate (e.g. for a human subject) is between 0.3-5 mg/kg body weight; or, in other embodiments, between 0.4-4 mg/kg body weight; or, in other embodiments, between 0.03-0.1 mg/kg body weight; or, in other embodiments, 0.03-0.09, 0.04-0.1, 0.04-0.09, 0.05-0.1, 0.05-0.09, 0.05-0.08, 0.055-0.08,,0.055-0.075, 0.06-0.075, 0.06-0.07, or 0.065-0.07 mg/kg body weight. In further embodiments, the pharmaceutical composition is indicated for administration up to three times daily, or, in more specific embodiments, once, twice, three times, or 1-2 times daily. In other embodiments, the pharmaceutical composition is indicated for administration 2-4, 2-3, or 3-4 times daily. In still further embodiments, the pharmaceutical composition further includes a lysine salt, or in other embodiments an arginine salt of suramin, at a dose of 0.3-0.6 mg/kg body weight; or, in other embodiments, 0.3-0.5, 0.35-0.45,.3-0.4, 0.4-0.5, or 0.4-0.6 mg/mg body weight. In certain embodiments, the daily dose does not exceed 0.6 mg/kg body weight, or, in other embodiments, 0.5, 0.4, 0.3, 0.2, or 0.1 mg/kg body weight. Those skilled in the art can readily calculate the doses of other norNOHA salts from the described doses of norNOHA acetate. Alternatively or in addition, the composition is administered for a maximum length of 90 days, or in other embodiments, 80 days, 70 days, 60 days, 50 days, 40 days, or 30 days.

In still other embodiments, the dose of norNOHA acetate (e.g. for a human subject) is between 0.5-1.5 mg/kg body weight, or, in other embodiments, between 0.75-1.5 mg/kg body weight. In still other embodiments, the dose is between 0.5-0.7, 0.6-0.8, 0.7-1.0, 0.8-1.2, 1-1.3, or 1.0-1.5 mg/kg body weight. In further embodiments, the pharmaceutical composition is indicated for administration up to three times daily, or, in more specific embodiments, once, twice, three times, or 1-2 times daily. In other embodiments, the pharmaceutical composition is indicated for administration 2-4, 2-3, or 3-4 times daily. In certain embodiments, the daily dose does not exceed 1.5 mg/kg body weight, or, in other embodiments, 1.4, 1.3, 1.2, 1.1, 1.0, 0.9, 0.8, or 0.7 mg/kg body weight. Those skilled in the art can readily calculate the doses of other norNOHA salts from the described doses of norNOHA acetate. Alternatively or in addition, the composition is administered for a maximum length of 90 days, or in other embodiments, 80 days, 70 days, 60 days, 50 days, 40 days, or 30 days.

In yet other embodiments, for short-term administration, 3.0-10.0 mg/kg body weight of norNOHA acetate (e.g. for a human subject) is administered. In other embodiments, the dose is 3.0-4.0, 3.5-4.5, 4.0-5.0, 4.5-5.5, 5-7, 6-8, or 7-10 mg/kg body weight. In other embodiments, the pharmaceutical compositions are indicated for short-term administration to an adult human (e.g., weighing an average of 70 kg.) at a dose of 30-100 mg of norNOHA acetate, or, in other embodiments, 20-350, 20-50, 40-100, 30-80, 40-80, 50-100, 75-125, 100-150, 125-175, 150-200, 175-225, 200-250, 225-300, 200-300, 225-325, 250-350 mg. Short-term administration, as used herein, may refer to administration for up to 2 weeks, or, in other embodiments, up to 10 days, or up to 8, 7, 6, 5, 4, 3, 2, or 1 day.

In yet other embodiments, administration of an arginase inhibitor (e.g. norNOHA acetate) is discontinued when a maximal tolerated body fluid concentration is reached. The maximal concentration may be, in some embodiments, 15 micromoles per liter (micromolar), or, in other embodiments, 20, 18, 12, 10, 8, 6, or 5 micromolar. Those skilled in the art can readily calculate the doses of other norNOHA salts from the described doses of norNOHA acetate. Optionally, administration may be recontinued when the body fluid concentration drops, e.g. to 50% of the maximal tolerated body fluid concentration.

The described dosage ranges of norNOHA acetate and suramin salts may be freely combined with one another.

In still other embodiments, a specific arginase inhibitor is utilized, such as N-hydroxy-guanidinium or N-hydroxy-nor-1-arginine; a boronic acid derivative, such as 2(S)-amino-6-boronohexanoic acid, and S-(2-boronoethyl)-1-cysteine; or an α-α-disubstituted amino acid-based arginase inhibitor [such as (R)-2-amino-6-borono-2-(2-(piperidin-1-yl)ethyl)hexanoic acid].

The described arginase inhibitors and dosage ranges thereof may be freely combined with the described suramin salts and dosages thereof.

In other embodiments, there is provided a method of treating a disease or disorder mentioned herein, comprising administering a herein-described composition, thereby treating the disease or disorder.

In other embodiments, there is a provided a pharmaceutical composition comprising a herein-described composition, for treatment of a herein-described disease or disorder.

In still other embodiments, there is provided a modified suramin formulation, e.g. as described herein, for use in a therapeutic method. In certain embodiments, the method is for treating a herein-described disease or disorder.

In other embodiments, there is provided use of a modified suramin formulation in the preparation of a medicament for a herein-described disease or disorder.

In other embodiments, there is provided an article of manufacture, comprising a packaging material and a pharmaceutical composition comprising a herein-described lysine or arginine salt of suramin, labeled as indicated for a herein-described disease or disorder.

Alternatively or in addition, each dose is administered in a series of 1, 2, 3, 4, 5, 6, 7, 8, 9, 10, 11, 12, 13, 14, 15, 1-10, 1-15, 1-20, 2-10, 2-15, 2-20, 3-20, 4-20, 5-20, 5-25, 5-30, 5-40, or 5-50 injections.

### Pharmaceutical Compositions

The described compounds can be administered as a part of a pharmaceutical composition, e.g., that further comprises one or more pharmaceutically acceptable carriers. Hereinafter, the term “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered compounds. Examples, without limitations, of carriers are propylene glycol, saline, emulsions and mixtures of organic solvents with water. In some embodiments, the pharmaceutical carrier is an aqueous solution of saline.

In other embodiments, compositions are provided herein that comprise the described compounds in combination with an excipient, non-limiting examples of which are an inert diluents and carriers such as a sugar or sugar alcohol, e.g. lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

The data obtained from these in vitro and cell culture assays and animal studies can be used in formulating a range of dosage for use in human. The dosage may vary depending upon the dosage form employed and the route of administration utilized. The exact formulation, route of administration and dosage can be, in some embodiments, chosen by the individual physician in view of the patient's condition.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

In other embodiments, the described compositions are suitably formulated as a pharmaceutical composition which can be suitably packaged as an article of manufacture. Such an article of manufacture comprises a packaging material which comprises a label describing a use in treating a disease or disorder or therapeutic indication that is mentioned herein. In other embodiments, a pharmaceutical agent is contained within the packaging material, wherein the pharmaceutical agent is effective for the treatment of a disorder or therapeutic indication that is mentioned herein. In some embodiments, the pharmaceutical composition is frozen.

It is clarified that each embodiment of the described compositions may be freely combined with each embodiment relating to a therapeutic method or pharmaceutical composition.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including suramin and derivatives thereof. In another aspect, the kits and articles of manufacture may comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1

A study is performed on female adult baboons found with abnormal colposcopy and histological verification of CIN I, CIN II and/or CIN III. Histological lesions will be scored using the criteria adapted from the CIN nomenclature system (Wright, T C., et al., 1994, A critical review of the morphologic classification system of preinvasive lesions of the cervix: the scientific basis for shifting the paradigm. Papillomavirus Rep. 5:175-182). Thus, common inclusion criteria into the study will involve diagnosis of grades 1-2 cervical intraepithelial neoplasia (CIN I-II), determined by histological evaluation of cervical biopsies from adult female baboons of reproductive age and beyond. Animals will be sedated during the procedures with ketamine/xylazine mixture at appropriate doses. Exclusion criteria will include the following: pregnant female baboons, breast-feeding and infections of the genitourinary system in the acute phase.

Baboons are randomized into three groups comprising of six animals each: group 1 [high dose, (n=6)] receives a combined formulation of Suramin-arginase inhibitor at a dose of 200 mg/day sublingually; group 2 [low dose, (n=6)] also receives a combined formulation of Suramin-arginase inhibitor at a dose of 100 mg/day sublingually; and group 3 [control group (n=6)] receives a control diluent. The course of treatment lasts 180 days without a break. Therapy is terminated in animals that display a complete CIN regression according to histological examination on day 90 of the study).

In the course of treatment for all three groups, cervical biopsies for histological assessment are done on day 0 (baseline cervical biopsies), day 30, day 90, and day 180 from the beginning of the therapy. All animals undergo a comprehensive survey during all sampling points, including clinical blood and urine analyses, cytology, and colposcopy with photography.

Treatment efficacy is assessed by the proportion of animals with complete CIN regression on the basis of the histological examination of biopsy specimens of the cervical lesions.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

What is claimed is:

1. A pharmaceutical composition comprising a lysine salt of suramin, formulated for sublingual administration.

2. The pharmaceutical composition of claim 1, indicated for administration to an adult human at a dose of 20-350 mg.

3. The pharmaceutical composition of claim 1, indicated for administration to a human at a dose of 15-50 mg.

4. The pharmaceutical composition of claim 2 or 3, indicated for administration up to three times daily.

5. The pharmaceutical composition of claim 1, indicated for administration to a human at a dose of 0.3-5.0 mg/kg body weight.

6. The pharmaceutical composition of claim 5, indicated for administration to a human at a dose of 0.3-0.6 mg/kg body weight.

7. The pharmaceutical composition of claim 5 or 6, indicated for administration up to three times daily.

8. The pharmaceutical composition of any of claims 1-7, for treatment of a virus selected from HIV/AIDS, Chikungunya, Ebolavirus, Hepatitis B virus, Zika virus, Hand Foot and Mouth Disease, and Coronavirus.

9. The pharmaceutical composition of claim 8, further comprising an antiretroviral compound.

10. The pharmaceutical composition of any of claims 1-7, for treatment of a Tuberculosis infection.

11. The pharmaceutical composition of any of claims 1-7, for treatment of an Onchocerciasis infection.

12. The pharmaceutical composition of any of claims 1-7, for treatment of a leukemia.

13. The pharmaceutical composition of claim 12, further comprising a chemotherapeutic agent.

14. The pharmaceutical composition of any of claims 1-7, for treatment of a prostate carcinoma.

15. The pharmaceutical composition of claim 14, further comprising a chemotherapeutic agent.

16. The pharmaceutical composition of any of claims 1-7, for treatment of a diabetic nephropathy.

17. The pharmaceutical composition of any of claims 1-7, for treatment of autism.

18. The pharmaceutical composition of any of claims 1-17, further comprising an arginase inhibitor

19. A pharmaceutical composition comprising an arginine salt of suramin, formulated for sublingual administration.

20. The pharmaceutical composition of claim 19, indicated for administration to an adult human at a dose of 20-350 mg.

21. The pharmaceutical composition of claim 19, indicated for administration to a human at a dose of 15-50 mg.

22. The pharmaceutical composition of claim 20 or 21, indicated for administration up to three times daily.

23. The pharmaceutical composition of claim 19, indicated for administration to a human at a dose of 0.3-5.0 mg/kg body weight. also add calculation per body fluid content.

24. The pharmaceutical composition of claim 23, indicated for administration to a human at a dose of 0.3-0.6 mg/kg body weight.

25. The pharmaceutical composition of claim 24, indicated for administration up to three times daily.

26. The pharmaceutical composition of any of claims 19-25, for treatment of a virus selected from HIV/AIDS, Chikungunya, Ebolavirus, Hepatitis B virus, Zika virus, Hand Foot and Mouth Disease, and Coronavirus.

27. The pharmaceutical composition of claim 26, further comprising an antiretroviral compound.

28. The pharmaceutical composition of any of claims 19-25, for treatment of a Tuberculosis infection.

29. The pharmaceutical composition of any of claims 19-25, for treatment of an Onchocerciasis infection.

30. The pharmaceutical composition of any of claims 19-25, for treatment of a leukemia.

31. The pharmaceutical composition of claim 30, further comprising a chemotherapeutic agent.

32. The pharmaceutical composition of any of claims 19-25, for treatment of a prostate carcinoma.

33. The pharmaceutical composition of claim 32, further comprising a chemotherapeutic agent.

34. The pharmaceutical composition of any of claims 19-25, for treatment of a diabetic nephropathy.

35. The pharmaceutical composition of any of claims 19-25, for treatment of autism.

36. The pharmaceutical composition of any of claims 19-35, further comprising an arginase inhibitor.
